(19) United States
(12) Patent Application Publication
(10) Pub. No.: US 2016/0317020 A1
(43) Pub. Date: Nov. 3, 2016

(54) PHASE GRADIENT OPTICAL COHERENCE TOMOGRAPHY ANGIOGRAPHY

(71) Applicants: Gangjun Liu, Portland, OR (US); David Huang, Portland, OR (US); Yali Jia, Portland, OR (US)

(72) Inventors: Gangjun Liu, Portland, OR (US); David Huang, Portland, OR (US); Yali Jia, Portland, OR (US)

(73) Assignee: OREGON HEALTH & SCIENCE UNIVERSITY, PORTLAND, OR (US)

(21) Appl. No.: 15/141,626

(22) Filed: Apr. 28, 2016

Related U.S. Application Data
(60) Provisional application No. 62/155,635, filed on May 1, 2015.

(51) Int. Cl. A61B 3/00; A61B 3/12; G01B 9/02

(52) U.S. Cl. CPC A61B 3/0025; G01B 9/02091; G01B 9/02004; G01B 9/0201; A61B 3/1233

(57) ABSTRACT

Disclosed are methods of imaging vascular flow using optical coherence tomography. The methods involve calculating an OCT phase difference and an OCT phase gradient from interference fringes acquired from B-scans. The methods can be implemented in a split-spectrum embodiment to enhance the signal to noise ratio of vascular flow images. The methods also obviate the need for correction of bulk motion and laser trigger jitter-induced phase artifacts.

### ACKNOWLEDGEMENT OF GOVERNMENT SUPPORT

This invention was made with the support of the United States government under the terms of grant number R01EY023285 awarded by the National Institutes of Health. The United States government has certain rights to this invention.

### FIELD

Generally, the field involves methods of imaging using optical coherence tomography. In particular, the field involves methods of visualizing blood flow using optical coherence tomography.

### BACKGROUND

Optical coherence tomography (OCT) is an imaging method that provides high-resolution, depth-resolved cross-sectional, and 3-dimensional (3D) images of biological tissue. Among its many applications, ocular imaging in particular has found widespread clinical use. New light source and detection techniques have resulted in improved OCT methodologies, including Fourier-domain OCT. Examples of Fourier-domain OCT include spectral (spectrometer-based) OCT and swept-source OCT (SS-OCT). These new methodologies provide superior sensitivity and imaging speed relative to older, time-domain OCT systems.

Another benefit of Fourier-domain OCT is that it allows the imaging of blood flow. Several approaches have been introduced for calculating blood flow from OCT images (collectively termed “OCT angiography”), but all of them generally work by computing the variation of OCT signal between consecutively acquired A-scans or B-scans. This signal variation is caused by moving blood cells within the image sample and can be used to highlight the presence of microvasculature against static surrounding tissue. For example, Doppler OCT images blood flow by evaluating phase differences between adjacent A-line scans. Doppler OCT is readily able to image and measure fast blood flow in larger blood vessels. However, it is unable to reliably distinguish the slower blood flow in smaller blood vessels from other types of biological motion in extravascular tissue. An additional challenge is that the use of Doppler OCT in imaging the retina involves detecting blood flow in vessels that are perpendicular or nearly perpendicular to the OCT beam. Detection of a Doppler shift signal depends on the beam incident angle, which is unavailable when blood flow is perpendicular to the OCT beam. As a result, detection of blood flow in smaller retinal vessels using techniques that do not rely on the beam incidence angle are necessary for retinal and choroidal OCT angiography.

Furthermore, the detection of blood flow by OCT angiography is vulnerable to motion artifacts. Such artifacts may arise due to sample or machine movement, for example, and can degrade the quality of OCT angiography data. In addition, SS-OCT systems are susceptible to phase artifacts that can be induced by trigger jitter. These trigger jitter artifacts reduce the precision of phase measurements and can introduce fixed pattern noise that degrades image quality. Thus, the removal or correction of such artifacts is essential for high quality OCT angiography imaging. However, current schemes for the correction or removal of these artifacts can be computationally cumbersome and produce imperfect results.

### SUMMARY

Disclosed are computer based methods of imaging vascular flow using optical coherence tomography (OCT), including, for example, swept source optical coherence tomography. The methods involve acquiring interference fringes from B-scans, transforming the interference fringes into depth encoded OCT data, and acquiring depth encoded OCT phases from the depth encoded OCT data. The methods further involve calculating an OCT phase difference using the depth encoded OCT data and further calculating an OCT phase gradient from the OCT phase difference. The interference fringes can be transformed into depth encoded OCT data by a fast Fourier transform, for example, by Equation 2 herein. The OCT phase gradient can be calculated by, for example, Equation 4. The methods can further involve acquiring a depth encoded OCT amplitude from the depth encoded OCT data and calculating a combination signal from the first depth encoded OCT amplitude and the OCT phase gradient. The combination signal can be calculated using, for example, Equation 5. The methods can further involve calculating a decorrelation of the phase gradient using the combination signal and another combination signal calculated from interference fringes from different B-scans as described above. The decorrelation can be calculated using, for example, Equation 6. This decorrelation of the phase gradient can be averaged with another decorrelation of the phase gradient acquired from interference fringes from different B-scans as described above. The methods can be implemented using split-spectrum techniques to enhance the signal to noise ratio of the resultant flow images. An aspect of the methods is that they allow the use of phase information to map flow images without the need for correction of phase artifacts caused by bulk motion or laser trigger jitter.

This Summary is provided to introduce a selection of concepts in a simplified form that are further described below in the Detailed Description. This Summary is not intended to identify key features or essential features of the disclosed subject matter, nor is it intended to be used to limit the scope of the disclosed subject matter. Furthermore, the disclosed subject matter is not limited to implementations that solve any or all disadvantages noted in any part of this disclosure.

### BRIEF DESCRIPTION OF THE SEVERAL VIEWS OF THE DRAWINGS

FIG. 1 is a flow diagram of a method used to calculate the phase gradient in a Fourier-domain OCT systems.

FIG. 2 is a flow diagram of a method used to calculate the combined amplitude phase gradient (CAPG) in a Fourier-domain OCT systems.

FIG. 3 is a flow diagram of a method used to calculate the decorrelation of amplitude combined phase gradient (CAPG) in a Fourier domain OCT system.

FIG. 4 is a flow diagram of a method used to calculate the split-spectrum phase-gradient angiography (SSPGA) in a Fourier domain OCT systems.

FIG. 5 is a flow diagram of a method used to calculate split-spectrum amplitude and phase-gradient angiography (SSAPGA) in a Fourier domain OCT systems.

FIG. 6 is a schematic of a spectral domain OCT system.

FIG. 7 is a schematic of a typical 1060 nm central wavelength swept source OCT system.

FIG. 8 is a schematic of a typical 1310 nm central wavelength swept source OCT system.

FIG. 9 is a set of six images that compare of different processing methods for the imaging of a flow phantom (1% intralipid solution in a 500 um inner diameter plastic tube) performed using a SSOCT system with a center wavelength of 1050 nm, a bandwidth of about 100 nm and a speed of 100 kHz. The images are processed from a single B-scan that contained 4096 A-scans and covered a lateral range of 2 mm. (a) Structural OCT cross section of a flow phantom (circular area) on top of static paper board (bottom). (b) and (c) show the flow images from phase-resolved color Doppler and phase-variance method. (d), (e) and (f) show the flow images obtained from PG, CAPG and amplitude decorrelation, respectively. Phase-resolved color Doppler and Doppler variance suffer from the trigger jitter induced phase instabilities as seen by flow noise in the static target on the bottom of (b) and (c). The PG (d), CAPG (e) and amplitude decor relation (f) do not show any flow noise.

FIG. 10 is a set of two 3 mm×3 mm OCT angiography images of human retinal circulation from SSADA (a) and SSAPGA (b) using a 70 kHz 840 nm spectral OCT system (RTVue-XR, Optovue). The imaging protocol consisted of a single volumetric scan covering a 3×3 mm scanning area centered on the fovea. In the fast transverse scanning direction, 304 A-scans were sampled to obtain a single B-scan. Two sequential B-scans were collected at each location for flow detection. In the slow transverse scanning direction, 304 locations were sampled. A scaling factor ρ of 2 was used for the SSAPGA in this realization and spit spectrum bands for both methods were 4. Here, the fovea avascular zone is chosen as the background region and the standard deviation in this region were found to be 128.2 for SSADA and 100.9 for SSAPGA. The average intensities outside of the fovea avascular zone are found to be 1533.0 for SSADA and 1566.1 for SSAPGA. So, an overall 29.8 percent SNR increase was found for SSAPGA as comparing to SSADA. The RMS contrast values were found to be 836.6 for SSADA and 1021.4 for SSAPGA. So an overall of 22.1 percent improvement was found for SSAPGA.

FIG. 11 is a flow diagram of a method used to calculate the decorrelation of amplitude combined phase gradient (CAPG) in a Fourier domain OCT system. Depth encoded complex OCT data are obtained from Fourier transform of the acquired fringes (may require addition calibration or dispersion compensation dependent on system used). The phase difference (or phase shift) between A-scans scans at the same location in sequential repeated B-scans is obtained. The axial gradient is calculated from the phase difference. This gradient is then combined with amplitude to calculate the decorrelation and then contrast.

FIG. 12 is a set of five images depicting the cross sectional structure of a healthy human retina and its associated microcirculation as calculated by four different methods.

(A) Structural OCT image. (B-E) OCT angiography images obtained by four different methods. The PGA shows high background noise (B). The three split-spectrum methods (SSPGA, SSADA and SSAPGA) show very similar results (C-E).

FIG. 13 is a set of four images showing the effect of averaging five adjacent frames of cross-sectional OCT angiography images to reduce noise, and a plot of the signal profile as a function of axial depth along the same A-scan of each of the four images. (A) PGA, (B) SSPGA, (C) SSADA, (D) SSAPGA, (E) Axial profiles along the yellow lines in (A)-(D).

FIG. 14 is a set of four 3 mm×3 mm OCT angiography images of human retinal circulation from as calculated by (A) PGA, (B) SSPGA, (C) SSADA, and (D) SSAPGA.

FIG. 15 is a set of cross sectional OCT intensity images as acquired from a healthy eye by a SS-OCT system and their corresponding OCT angiography images determined by phase difference and SSPGA. (A, D) Cross-sectional OCT intensity images. (B, C, E, F) OCT angiography images. The phase difference images (B, E) show vertical line artifacts due to the depth dependent phase difference induced by the triggering jitter and bulk motion. The axial gradient of the phase difference image shows clear vessel boundaries (C, F).

FIG. 16 is a set of three images showing the en face projection OCT angiography of human disc and retinal circulation acquired with a SS-OCT system. (A) SSPGA, (B) SSADA, (C) SSAPGA methods were used to process the scan data.

FIG. 17 is a schematic of a system for processing OCT angiography data in accordance with the disclosure.

FIG. 18 is an example of a computing system in accordance with the disclosure.

### DETAILED DESCRIPTION

In the following detailed description, reference is made to the accompanying drawings which form a part hereof, and in which are shown by way of illustration embodiments that can be practiced. It is to be understood that other embodiments can be utilized and structural or logical changes can be made without departing from the scope. Therefore, the following detailed description is not to be taken in a limiting sense, and the scope of embodiments is defined by the appended claims and their equivalents.

Various operations can be described as multiple discrete operations in turn, in a manner that can be helpful in understanding embodiments; however, the order of description should not be construed to imply that these operations are order dependent.

The description may use the terms “embodiment” or “embodiments,” which may each refer to one or more of the same or different embodiments. Furthermore, the terms “comprising,” “including,” “having,” and the like, as used with respect to embodiments, are synonymous.

In various embodiments, structure and/or flow information of a sample can be obtained using OCT (structure) and OCT angiography (flow) imaging-based on the detection of spectral interference. Such imaging can be two-dimensional (2-D) or three-dimensional (3-D), depending on the application. Structural imaging can be of an extended depth range relative to prior art methods, and flow imaging can be performed in real time. One or both of structural imaging and flow imaging as disclosed herein can be enlisted for producing 2-D or 3-D images.

Unless otherwise noted or explained, all technical and scientific terms used herein are used according to conventional usage and have the same meaning as commonly understood by one of ordinary skill in the art which the disclosure belongs. Although methods, systems, and apparatuses/materials similar or equivalent to those described herein can be used in the practice or testing of the present disclosure, suitable methods, systems, and apparatuses/materials are described below.

All publications, patent applications, patents, and other references mentioned herein are incorporated by reference in their entirety. In case of conflict, the present specification, including explanation of terms, will control. In addition, the methods, systems, apparatuses, materials, and examples are illustrative only and not intended to be limiting.

Several OCT-based techniques have been successfully developed to image microvascular networks in human eyes in vivo. Algorithms for use in OCT angiography have been developed. These algorithms have been based on measurements of intensity/amplitude values, phase values or a combination of both phase and amplitude values (complex values) (Jia Y and Wang R K, J Biophotonics 4, 57-63 (2011); Mariampillai A et al, Opt Lett 33, 1530-1532 (2008); Liu G et al, Opt Express 19, 11429-11440 (2011); Jia Y et al, Opt Express 20, 4710-4725 (2012); Yu L and Chen Z, J Biomed Opt 15, 016029 (2010); Kim D Y et al, Biomed Opt Express 2, 1504-1513 (2011); Yang V X et al, Opto Commun 208, 209-214 (2002); Park B H et at Opt Express 11, 782-793 (2003); Nam A S et al, Biomed Opt Express 5, 3822-3832 (2014); and Enfield J et al, Biomed Opt Express 2, 1184-1193 (2011); all of which are incorporated by reference herein). One such example is optical microangiography (OMAG), which can resolve the fine vasculature in both retinal and choroid layers. OMAG uses a modified Hilbert transform to separate the scattering signals from static and moving scatters. By applying the OMAG algorithm along the slow scanning axis, high sensitivity imaging of capillary flow can be achieved.

However, because of the high sensitivity of OMAG the removal of bulk-motion must be performed precisely. This is currently done by resolving the Doppler phase shift. A major drawback of this methodology is that it is susceptible to artifacts from system or biological phase instability.

Other related methods such as phase variance and Doppler variance have been developed to detect small phase variations from microvascular flow. These methods are improvements on earlier techniques in that they can detect both transverse and axial flow and have been successful in visualizing retinal and choroidal microvascular networks. However, similar to OMAG, these phase-based methods also require precise removal of background Doppler phase shifts resulting from the axial movement of bulk tissue (Yang et al 2002 supra and Swanson E A et al, Optics Letters 18, 1864-1866 (1993); incorporated by reference herein). Artifacts can also be introduced by phase noise in the OCT system and transverse tissue motion, and these also need to be removed.

Most of the methods described above have been based on spectral OCT. Spectral OCT provides high phase stability which in turn allows for the evaluation of phase shifts and differentiation of the phase contrast resulting from blood flow. In contrast, swept-source OCT introduces phase variation through cycle-to-cycle tuning and timing variability (Zhang J and Chen Z, Opt Express 13, 7449-7457 (2005) and Vakoc B et al, Opt Express 13, 5483-5493 (2005); both of which are incorporated by reference herein). Therefore, phase-based angiography is noisier than spectral OCT. Consequently, the use of phase-based angiography methods on swept-source OCT systems requires complex methods to reduce system phase noise.

All that said, swept-source OCT offers several advantages over spectral OCT. It has a longer imaging range, results in less depth-dependent signal roll-off, and results in less motion-induced signal loss due to fringe washout. An amplitude-based OCT signal analysis has been demonstrated, but algorithms that exploit complex values can provide enhanced image contrast and higher detection sensitivity by combining contrast from both phase and amplitude values. A methodology that provides high phase stability that is adapted for swept source OCT would be a significant advance over what is currently present in the art.

Described herein is a phase gradient (PG) method which allows the use of phase information to map the microvasculature in tissue without the need for (a) correction of bulk motion of tissue and (b) correction of laser trigger jitter-induced phase artifacts using a swept-source OCT system. Combining PG with amplitude allows the use of contrast from both the phase and amplitude information and provides better OCT angiography images with higher image contrast and higher sensitivity. The split spectrum method is used to improve the signal-to-noise ratio (SNR) of flow detection, similar to the approach used in the split-spectrum amplitude decorrelation angiography (SSADA) algorithm (Jia Y et al 2012 supra).

The following explanation of specific terms is provided:

A-scan (or A-line): A reflectivity profile that contains information about the spatial dimensions and/or location of structures with an item of interest. Such a scan can also be referred to as an axial depth scan.

Autocorrelation: A cross-correlation of a signal with itself; the similarity between observations as a function of the time separation between them. For example, autocorrelation can be used to find repeating patterns, such as the presence of a periodic signal which has been buried under noise, or used to identify the missing fundamental frequency in a signal implied by its harmonic frequencies.

B-scan: A cross-sectional tomograph that results from the lateral combination of a series of A-scans (described above).

Bulk movement: any movement of a tissue being imaged by OCT that is not red blood cell movement within a blood vessel of the tissue.

Cross-correlation: A measure of similarity of two waveforms as a function of a time-lag applied to one of the waveforms.

Decorrelation: A process that is used to reduce autocorrelation within a signal, or cross-correlation within a set of signals, while preserving other aspects of the signal. For example, decorrelation can be used to enhance differences found in each pixel of an image. A measure of a lack of correlation or similarity between corresponding pixels in two images can also describe decorrelation. The end result of a decorrelation process is that faint information within a signal may be enhanced to bring out (e.g., present) subtle differences that may be meaningful. For example, one can calculate decorrelation to find a difference between images.

Sample: Any part of any subject that comprises vasculature that is capable of being imaged by OCT. In one example, structures of the eye are imaged, including the retina and other structures of the eye. In particular, the vasculature of the eye is imaged by OCT.

OCT angiography visualizes tissue vasculature. However, this process is complicated by the fact that any bulk movement of the sample or between the imaging system and the sample will result in a phase shift that must be corrected for proper imaging. For example, in the case of imaging of structures of the eye, any involuntary eye movement resulting from muscle twitch or any bulk movement caused by, for example, breathing can result in a phase shift. In addition, one or more components of the system can be moved. For example, these components can be moved accidentally by the operator (Yang V X et al 2002 supra; Swanson E A et al, 1993 supra; and Yazdanfar S et al, Opt Lett 25, 1448-1450 (2000); incorporated by reference herein.)

Phase shift signals induced by bulk movement are mixed with the phase shift signals resulting from red blood cell (RBC) movement in blood vessel. So the actual phase shift (Δφ(x,z)) received is a combination of these two types of phase shift—this combination is expressed by Equation 1 below.

Δφ(x,z)=Δφᵥ(x,z)+Δφ_{b}(x) Equation 1

where x is the lateral location, z is the depth location, Δφᵥ is the phase resulting from by RBC movement inside the blood vessel and Δφ_{b} is the phase resulting from bulk motion. The bulk phase Δφ_{b} is typically a global term which is independent of depth.

Swept source OCT devices can use a mechanical device to perform a wavelength sweep. As a result, there can be uncertainty in trigger timing such that the starting time of spectral interferogram acquisition changes from cycle to cycle in terms of the wave number (k). This uncertainty is known in the art as the ‘trigger jitter’. Accounting for phase uncertainty resulting from trigger jitter can be performed as follows: If there is a relative k shift of δ between two received interferograms called A1(k) & A2(k), then A2(k)=A1(k+δΔk), where Δk is the sampling wavenumber interval in a Fourier domain. A2(k) and A1(k) differ only by the phase term of Equation 2 below.

F(A2(k))=exp(j2πzδ/N)F(A1(k)) Equation 2

where F( ) is the Fourier transform and z is the depth position of the A-scan. Therefore, the actual phase difference (Δφ(x,z)) obtained is a sum of (a) the actual phase difference (Δφᵥ(x,z)) induced by flow, (b) the phase difference induced by bulk movement (Δφ_{b}(x)) and (c) phase uncertainty induced by trigger jitter (2πδ(x)z/N). So the actual phase difference can be calculated by Equation 3 below.

Δφ(x,z)=Δφᵥ(x,z)+Δφ_{b}(x)+2πδ(x)z/N Equation 3

The bulk movement phase difference Δφ_{b}(x) is typically removed by the following methods: since the blood vessels are usually small relative to the whole imaging depth, an assumption is made that the vessels should encompass fewer axial pixels than non-vessel tissue. Based on this assumption, a simple median/mean value search along the depth direction at the specific lateral location will reveal the phase difference due to bulk movement (White B R et al, Optics Express 25, 3490-3497 (2003); incorporated by reference herein). Histogram based maximum bin searching methods can also be used (Yu L and Chen Z, 2010 supra; Kim D Y et al, 2011 supra; Yang V X et al, 2002 supra; Makita S et al, Opt Express 14, 7821-7840 (2006); Liu G et al, Opt Express 19, 3657-3666 (2011); and An L and Wang R K, Opt Express 16, 11438-11452 (2008); all of which are incorporated by reference herein). The above methods are less effective in regions with relatively large vessels or with strong vessel shadows, so additional methods may be needed (Rao B et al, J Biomed Opt 13, 040505 (2008); incorporated by reference herein) in imaging such regions.

Trigger jitter induced phase differences in swept source OCT systems can be removed by manipulating the hardware, the software, or a combination of both. For example, a trigger signal with a fiber Bragg grating (FBG) can be used to remove trigger jitter at the digitizer (Hendargo H C et al, Biomed Opt Express 2, 2175-2188 (2011); incorporated by reference herein). Alternatively, a method to resample the interference fringe to the exact wavenumber space using a simultaneously recorded calibration interference signal has also been described (Braaf B et al, Opt Express 19, 20886-20903 (2011); incorporated by reference herein). Furthermore, a narrow band FBG can be used to produce a reference dip in acquired interferograms, followed by a shifting of the interferograms (Choi W et al, Opt Lett 38, 338-340 (2013); incorporated by reference herein). Finally, a method that aligns interferograms by minimizing fixed pattern noise intensity has also been described (Liu G et al, Optics Express 23, 9824-9834 (2015); incorporated by reference herein).

Disclosed herein is a method termed phase gradient imaging. This method takes the axial gradient of the phase difference Δφ(x,z) described in Equation 3 and from it, calculates an axial phase gradient (PG) as shown in Equation 4 below.

$$PG ( x , z ) = \frac{d ( Δφ ( x , z ) )}{d z} = \frac{d ( {Δφ}_{v} ( x , z ) )}{d z} + r ( x ) \qquad Equation 4$$

Where r(x)=2πδ(x)/N. The use of PG eliminates the phase difference due to bulk movement and reduces the phase uncertainty resulting from trigger jitter induced to a constant value r(x). The remaining term

$$\frac{d ( {Δφ}_{v} ( x , z ) )}{d z}$$

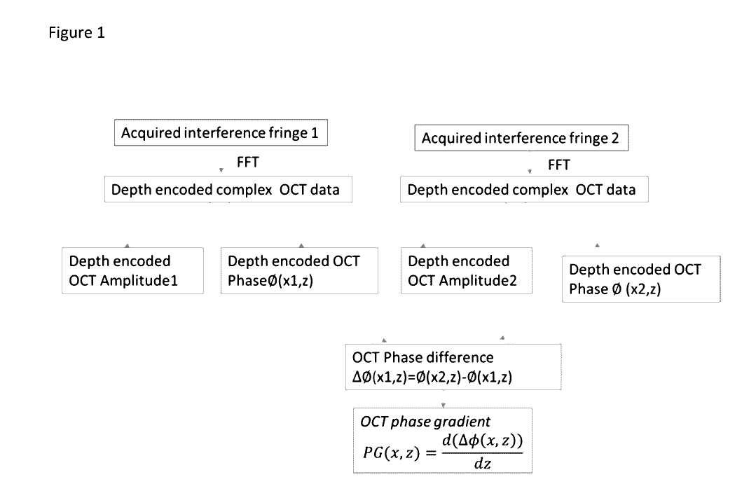

is the axial gradient of the phase difference induced by the blood flow. The r(x) value is typically very small. For N=1024 and δ(x)=1, r(x) is around 6 milliradian per depth pixel. FIG. 1 summarizes the basic method used in calculating the phase gradient using a typical Fourier domain OCT system.

The PG value can be combined with the OCT signal amplitude to enhance flow contrast. This combination can be performed by replacing an OCT signal phase with PG. For example, if the OCT signal amplitude is A(x, z), then a new combination signal \tilde{S} that combines amplitude (A) with PG (CAPG) can be determined using Equation 5 below.

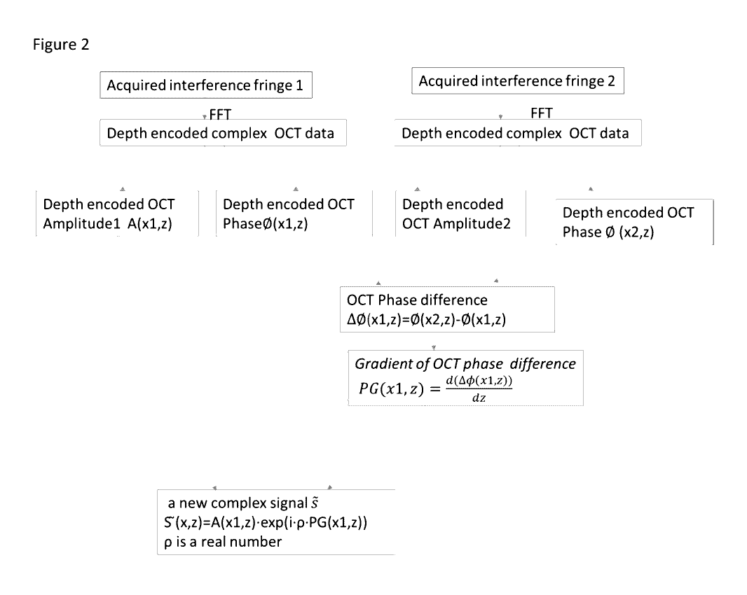

\tilde{S} (x,z)=A(x,z)·exp(i·ρ·PG(x,z)) Equation 5

where ρ is a unitless scaling factor used to control the phase gradient contribution. The value of ρ is chosen by the user. In the examples presented herein, p=2 is used. FIG. 2 shows the shows the typical steps to calculate the amplitude combined phase gradient (CAPG) in typical Fourier domain OCT systems. FIG. 2 summarizes the basic method used in calculating the CAPG using a typical Fourier domain OCT system.

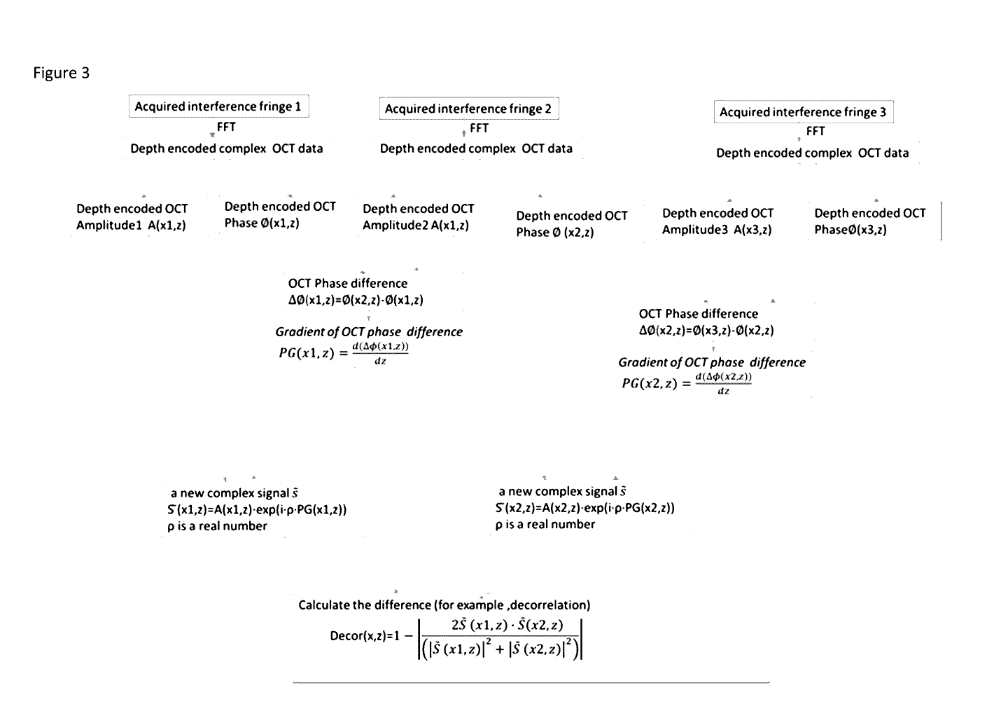

Flow contrast can be obtained by calculating the decorrelation of \tilde{S} between adjacent A-scans and B-scans using Equation 6 below:

$$Decor ( x , z ) = 1 - \vert {\sum }_{x = 1}^{X} \frac{2 \tilde{S} ( x , z ) \cdot \tilde{S} ( x + 1 , z )}{( {\vert \tilde{S} ( x , z ) \vert}^{2} + {\vert \tilde{S} ( x + 1 , z ) \vert}^{2} )} \vert \qquad Equation 6$$

where X is the number of averaging frames. FIG. 3 shows the typical steps to calculate the decorrelation of the amplitude combined phase gradient (CAPG) in typical Fourier domain OCT systems.

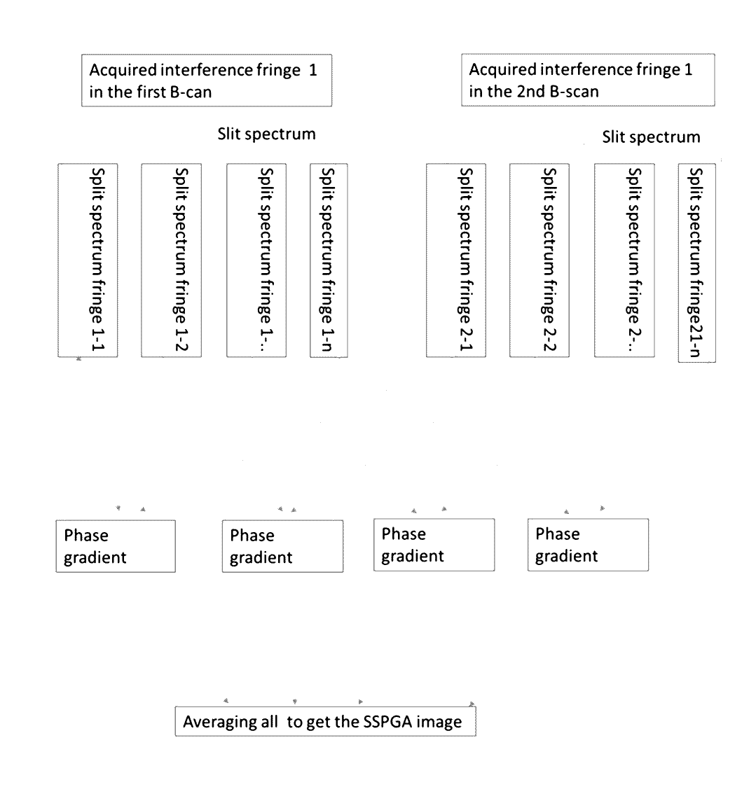

A split spectrum method similar to the previously published split-spectrum amplitude decorrelation angiography (SSADA) algorithm (Jia Y et al 2012 supra) is used to further improve the signal-to-noise ratio (SNR) of flow detection for the methods described herein. The combined algorithm is called split-spectrum amplitude and phase-gradient angiography (SSAPGA) or split-spectrum phase-gradient angiography (SSPGA). FIG. 4 shows an example of the typical steps to calculate SSPGA and SSAPGA.

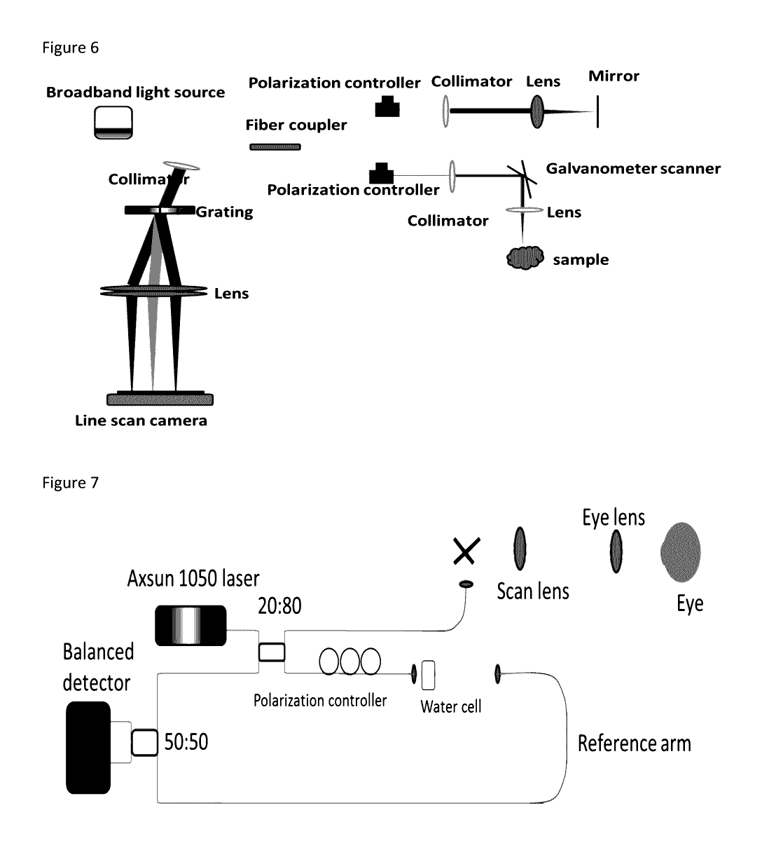

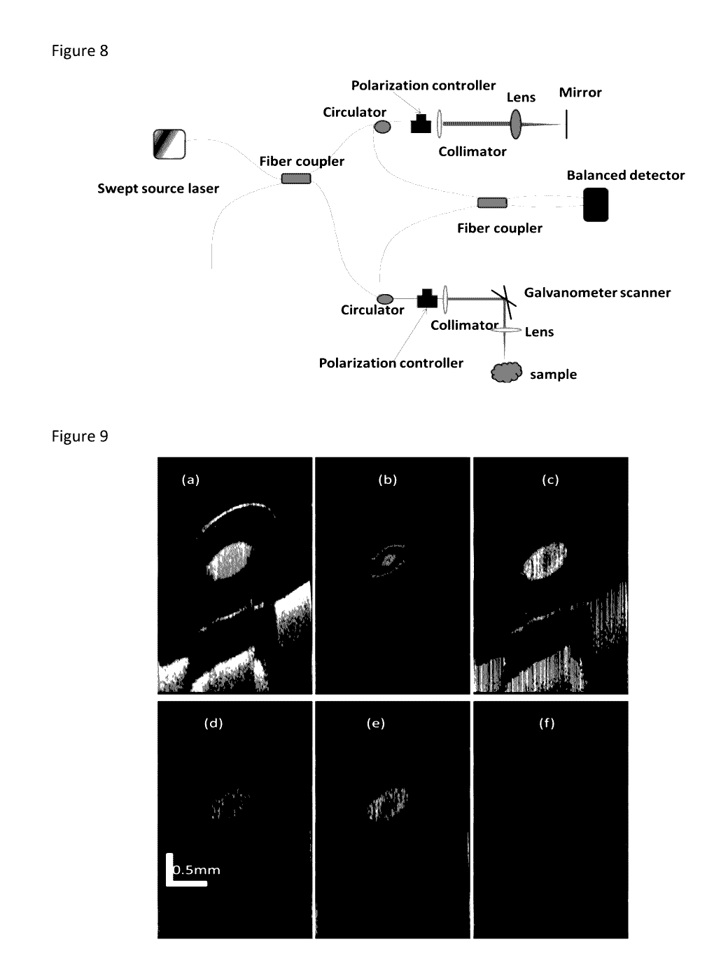

The methods proposed in this invention can be performed using either time domain OCT systems or Fourier domain OCT systems. Use of Fourier domain OCT systems are exemplified here. FIGS. 6, 7 and 8 show the typical setups of a spectral domain OCT system, a typical 1060 nm central wavelength swept source OCT system and of a typical 1310 nm central wavelength swept source OCT system. The proposed methods can be applied to adjacent A-scan interferograms or to adjacent B-scan interferograms. For an OCT angiography application, a B-M scanning mode can be used. In such a B-M scanning mode embodiments, sequential B-scans are repeated at the same location and the disclosed algorithms are applied to these sequential B-scans.

### EXAMPLES

### Example 1

### Results of Imaging a Flow Phantom

A custom built swept source OCT system (SSOCT) was used to test the results of the image correction methods described herein in a flow phantom study. The SSOCT system used has a center wavelength of 1050 nm with a bandwidth of around 100 nm and operating speed of 100 kHz. The SSOCT system has an axial resolution of 7.1 μm in air and a lateral resolution of 19 μm. A 500 um inner diameter plastic tube was filled with 1% intralipid solution and pumped by a syringe pump with a pump speed of 100 ul/min. A piece of paper board was put below the plastic tube to simulate static tissue. FIG. 9 shows the OCT image and flow images obtained from difference methods. The images are acquired with a single B-scan that contained 4096 A-scans and covered a lateral range of 2 mm. FIG. 9(a) shows the structural OCT cross section of the flow phantom (circular area) resting atop a static target. FIGS. 9(b) and 9(c) show the flow images resulting from phase-resolved color Doppler and phase-variance method. FIGS. 9(d), 9(e) and 9 (f) show the flow images obtained from PG, CAPG and amplitude decorrelation, respectively. The PG and CAPG algorithms based on Equations 4 and 6 herein were used between adjacent A-scans to generate FIGS. 9(d) and 9(e). A scaling number ρ of 2 was used in this example. The amplitude decorrelation image in FIG. 9(f) was obtained using the SSADA algorithm (Jia Y et al 2012 supra).

### Example 2

### Results of Imaging In Vitro

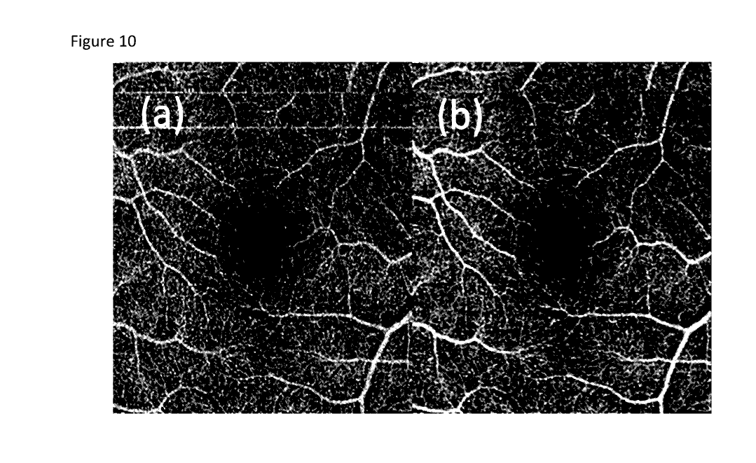

The methods described herein were tested for clinical retinal imaging using a 70 kHz 840 nm spectral domain OCT system (RTVue-XR, Optovue) on a normal human subject (FIG. 10). The imaging protocol consisted of a single volumetric scan covering a 3×3 mm scanning area centered on the fovea. In the fast transverse scanning direction, 304 A-scans were sampled to obtain a single B-scan. Two sequential B-scans were collected at each location for flow detection. In the slow transverse scanning direction, 304 locations were sampled. SSAPGA with 4 split bands was used to process the data.

The light source was split into four different narrow band spectra using Gaussian Windows. The narrow band spectra had different central wavelengths but the same FWHM value. The narrow band spectra were processed separately using typical spectral domain OCT data processing methods. These methods include numerical interpolation for wavelength calibration, dispersion compensation, and a fast Fourier transform (FFT). The phase term of the complex OCT signal after the FFT was used to obtain the PG signal using Equation (4). This PG signal was then combined with the amplitude to obtain the CAPG signal \tilde{S} using Equation (5). SSAPGA was obtained by calculating the decorrelation of \tilde{S} among the repeated B-scans using Equation (6). Here, X in Equation (6) is the number of split-bands (4 in this case). As above, a scaling number ρ=2 was used. Results from SSADA (using 4 split bands) were also obtained for comparison. The SSADA algorithm used similar parameters and processing procedures as SSAPGA except that the amplitude decorrelation was calculated instead of the complex value \tilde{S} decorrelation.

The 3D data was separated into retinal and choroidal regions with the dividing boundary set at the retina pigment epithelium (RPE) layer. The region above the RPE was defined as the retina. FIG. 10 shows the average intensity en face projection angiography images for human retinal vasculature obtained using SSADA (FIG. 10(a)) and using SSAPGA (FIG. 10(b)). Two parameters, the image signal to noise ratio (SNR) and the image contrast, were used to quantitatively analyze the images. SNR for flow detection was calculated by using the foveal avascular zone as the noise region. The standard deviation in the foveal avascular zone was found to be 128.2 for SSADA and 100.9 for SSAPGA. The average intensities outside of the fovea avascular zone were found to be 1533.0 for SSADA and 1566.1 for SSAPGA. An overall 29.8 percent SNR increase was found for SSAPGA compared to SSADA.

Root mean square (RMS) contrast was used to quantify image contrast for comparison purposes. RMS contrast was defined as the standard deviation of the pixel intensities (Peli E, J Opt Soc Am A 7, 2032-2040 (1990); incorporated by reference herein) as described in Equation 7 below.

$${C}_{rms} = \sqrt{\frac{1}{XY} {\sum }_{x = 1}^{X} {\sum }_{y = 1}^{Y} {[ V ( x , y ) - \overline{V} ]}^{2} \overline{V}} \qquad Equation 7$$

where X and Y are the number of pixels in the two dimensional image, V(x,y) are the value for the pixel at the (x,y) location in the image and V is the average value of all pixels in the image. The RMS contrast values were found to be 836.6 for SSADA and 1021.4 for SSAPGA, an overall improvement of 22.1 percent when using SSAPGA.

### Example 3

### Comparison of OCT Angiography Methods

Background: Fluorescein angiography (FA), the current standard of care for retinal vasculature imaging, requires intravenous injection of a dye that can cause nausea in 10% of patients and even anaphylaxis or death in rare cases. Optical coherence tomography angiography (OCTA), a recently developed clinical retinal and choroidal imaging technique, can obtain high-quality, high-contrast angiograms quickly without dye contrast. Instead, OCTA detects blood flow by computing the variation of OCT signal between consecutive cross-sectional images (B-frames) taken at the same location. This variation is caused by moving blood cells and provides microvasculature contrast against static retinal tissue. OCTA does not require a hardware modification, allowing it to be implemented on most currently available, high scan speed OCT systems.

Several OCTA algorithms have been proposed for both spectral-domain OCT (SD-OCT) and swept source OCT (SS-OCT). These methods compute the variation of OCT signal in terms of amplitude/intensity, phase, or complex values (combination of phase and amplitude) as described above. Amplitude/intensity based OCTA algorithms include speckle variance, intensity-based Doppler variance, intensity-based optical microangiography (OMAG), cross-correlation mapping, and split-spectrum amplitude decorrelation angiography (SSADA). These algorithms use variance, decorrelation, cross-correlation, or absolute difference of OCT signal amplitude/intensity between consecutive B-scans to map the microvasculature. The amplitude/intensity based OCTA methods have been shown to be less vulnerable to motion artifacts (Mariampillai et al 2008 supra; Liu G et al, Biomed Opt Express 3, 2669-2680 (2012); Jonathan E et al, J Biophotonics 4, 583-587 (2010); Jia et al 2012 supra; all of which are incorporated by reference herein). In contrast, phase and complex OCTA algorithms, such as Doppler variance, phase variance, and OMAG, require the removal or correction of motion artifacts (Yu L and Chen Z 2010 supra; Kim D Y et al 2011 supra; Liu G et al 2012 supra; Wang R K and An L, Opt Express 17, 8926-8940 (2009). The removal of bulk motion phase artifact (induced by sample or machine movement) is essential for high quality angiography imaging, especially for in-vivo applications (Yang V X et al 2002 supra; Swanson E A et al 1993 supra). Additional phase artifacts may also be induced by the laser trigger jitter of SS-OCT systems (Zhang J and Chen Z 2005 supra; and Vakoc B et al 2005 supra). Unlike amplitude/intensity based methods, phase and complex OCTA algorithms require either software or hardware methods to reduce these trigger jitter induced phase artifacts.

In this third Example, phase gradient angiography (PGA) for OCTA application is described. PGA is capable of mapping retinal microvasculature without the correction of bulk motion and laser trigger jitter induced phase artifacts. Several extensions of the PGA method are also introduced in this Example. It is demonstrated that that PGA can be combined with signal amplitude to improve performance. Furthermore, the split-spectrum technique can be applied to increase the signal to noise ratio (SNR). Exemplary formulations for split-spectrum phase gradient angiography (SSPGA) and split-spectrum amplitude and phase-gradient angiography (SSAPGA) are described herein. Quantitative comparison between PGA, SSADA, SSPGA, and SSAPGA methods applied in-vivo, using a 70 kHz SD-OCT system and a 200 kHz SS-OCT system.

Methods: The goal of phase OCTA methods is to extract only the phase shift induced by red blood cell (RBC) movement in blood vessels. For in-vivo applications, any relatively small movement between the sample and OCT system introduces additional phase shifts. This includes sample movement caused by involuntary movement of the participant (such as breathing), environmental vibrations, or accidental movement of the machine by the operator. Additional phase noise is mixed with phase shift signal originating from RBC movement. In the following paragraphs, features of the different phase shifts are analyzed.

To simplify the presentation of material in this Example, only B-scans acquired at a single location are analyzed (i.e., the analysis is limited to two dimensions). Three-dimensional analysis can be done by extending the analysis to all repeated B-scan locations. The complex OCT data signal at lateral x, axial location z, and time t in a B-scan is shown in Equation 8 below.

S(x,z,t)=A(x,z,t)·exp(i·φ(x,z,t)) Equation 8

where A is the signal amplitude and φ is the phase. Because repeated B-scan protocol is used, the complex OCT data signal for the same location (x,z) from the repeated B-scan is represented as shown in Equation 9 below.

S(x,z,t+Δt)=A(x,z,t+Δt)·exp(i·φ(x,z,t+Δt)) Equation 9

where Δt is the time interval between the two repeated B-scans. The phase difference at location (x, z) between the two repeated B-scans can be calculated directly by subtraction as shown in Equation 10 below.

Δφ_{E}(x,z,t)=φ(x,z,t+Δt)−φ(x,z,t) Equation 10

However, Equation 10 may not obtain the actual phase shift induced by RBC movement inside the vessel. Instead, it also includes phase noise and phase artifact originating from sample movement and equipment vibration as described previously. To separate the phase shift induced by RBC movement from phase artifact and phase noise, we rewrite the above Equation 10 into the form of Equation 11 below.

Δφ_{E}(x,z,t)=Δφᵥ(x,z,t)+Δφₐ(x,z,t)+Δφₙ(x,z,t) Equation 11

where Δφᵥ is the phase induced by RBC movement, Δφₐ is phase artifact and Δφₙ is the phase noise.

In SD-OCT, the main source of Δφₐ is the bulk phase (Δφ_{b}) caused by the participant's involuntary movement. The bulk phase Δφ_{b} is typically independent of depth z and is only a function of x. For SS-OCT systems, another source of phase artifact is caused by the mechanical scanning device used for wavelength sweeping. This mechanical instability results in uncertainty with trigger timing so that the starting point of the spectral interferogram acquisition changes from cycle to cycle in wavenumber (k) space (Vakoc B et al 2005 supra). If there is relative k shift of δ between two acquired interferograms G1(k) and G2(k), this shift can be represented as in Equation 12 below.

G2(k)=G1(k+δΔk) Equation 12

where Δk is the sampling wavenumber interval.

The Fourier transformations of G1(k) and G2(k) only differ on the phase term (Liu G et al, Opt Express 23, 9824-9834 (2015); incorporated by reference herein) as shown in Equation 13 below.

$$F ( G 2 ( k ) ) = exp ( j \frac{2 π z δ}{N} ) F ( G 1 ( k ) ) \qquad Equation 13$$

where F( ) means Fourier transform and N is the number of data points in an A-scan. Therefore, in a SS-OCT system, an additional depth dependent phase term of 2πzδ/N may exist. This phase will contribute as another kind of artifact to the phase shift obtained from Equation 10. Consequently, the phase shift (Δφ_{E}) may be the sum of actual phase difference (Δφᵥ(x,z)) induced by flow, bulk phase (Δφ_{b}) induced by sample movement, and trigger jitter-induced depth dependent phase (2πδz/N) and phase noise, as shown in Equation 14 below.

Δφ_{E}(x,z,t)=Δφᵥ(x,z,t)+Δφ_{b}(x,t)+2πδ(t)z/N+Δφₙ(x,z,t) Equation 14

To eliminate these two kinds of phase artifacts, separate schemes have to been used. Bulk phase term Δφ_{b} is typically removed using pure numerical methods. Since the blood vessels are usually inside tissue and are relatively small, the vessels make up fewer axial pixels than non-vessel tissue. Based on this assumption, a simple median/mean value search along the depth direction will find the bulk phase term (Yang V X et al 2002 supra; White B R et al 2003 supra). By repeating this step for all lateral locations, the bulk phase can be obtained for all the locations. In recent years, a histogram based maximum bin searching method has been shown to give better performance (Yu L and Chen Z 2010 supra; Kim D Y et al, 2011 supra; Yang V X et al, 2002 supra; Makita S et al, 2006 supra; Liu G et al, 2011 supra; and An L and Wang R K, 2008 supra). Although effective, in regions with relatively large vessels or strong vessel shadows, these methods will fail to generate the correct bulk phase and can require additional correction steps (Rao B et al, 2008 supra).

For SS-OCT systems, corrections of phase instability induced by trigger jitter have been demonstrated with hardware and software methods. Hendargo et al. demonstrated a way to reduce the trigger jitter by generating the A-scan trigger signal with a narrow linewidth fiber Brag grating (FBG) (Hendargo H C et al, 2011 supra). Braaf et al. proposed a method to resample the interference fringe to the exact wavenumber space using a simultaneously recorded calibration interference signal (Braaf B et al, 2011 supra). Choi et al. improved the SS-OCT phase stability by shifting the interferograms with a reference dip produced from a narrow linewidth FBG (Choi W et al, 2013 supra). A method that aligns the interferograms by minimizing fixed pattern noise intensity has also been described (Liu G et al, 2015 supra). However, these methods either increase the system complexity and cost, or they increase the computation time.

In this Example, a PGA method for OCTA application is described. By taking the axial gradient of the phase difference Δφ_{E}(x,z,t) in Equation 14, axial phase gradient can be obtained as shown in Equation 15 below.

$$\begin{matrix}PGA ( x , z , t ) = \frac{d ( {Δφ}_{E} ( x , z , t ) )}{d z} \\ = \frac{d ( {Δφ}_{v} ( x , z , t ) )}{d z} + \frac{2 π δ ( t )}{N} + \\ {Δφ}_{n} ( x , z , t )\end{matrix} \qquad Equation 15$$

Note that Δφ_{b}(x,t) is not dependent on the depth z, so its axial gradient is zero. Thus, the phase gradient calculated by Equation 15 will eliminate the bulk phase. The trigger jitter induced phase is a linear function of depth z and its axial gradient is a constant value: 2πδ(t)/N. The constant value, 2πδ(t)/N is typically very small. For example, when N=1024 and δ=1, the value is around 6 milliradians per depth pixel. This value is much smaller than the phase noise Δφₙ which is typically produced by galvanometer scanners (Braaf K et al, Opt Express 20, 20516-20534 (2012); incorporated by reference herein). The remaining term

$$\frac{d ( {Δφ}_{v} ( x , z ) )}{d z}$$

in Equation 15 is the axial gradient of the phase shift induced by RBC in the blood vessels. Phase gradient can be used to image the vessels alone. It may also be combined with OCT signal amplitude/intensity to enhance the flow contrast.

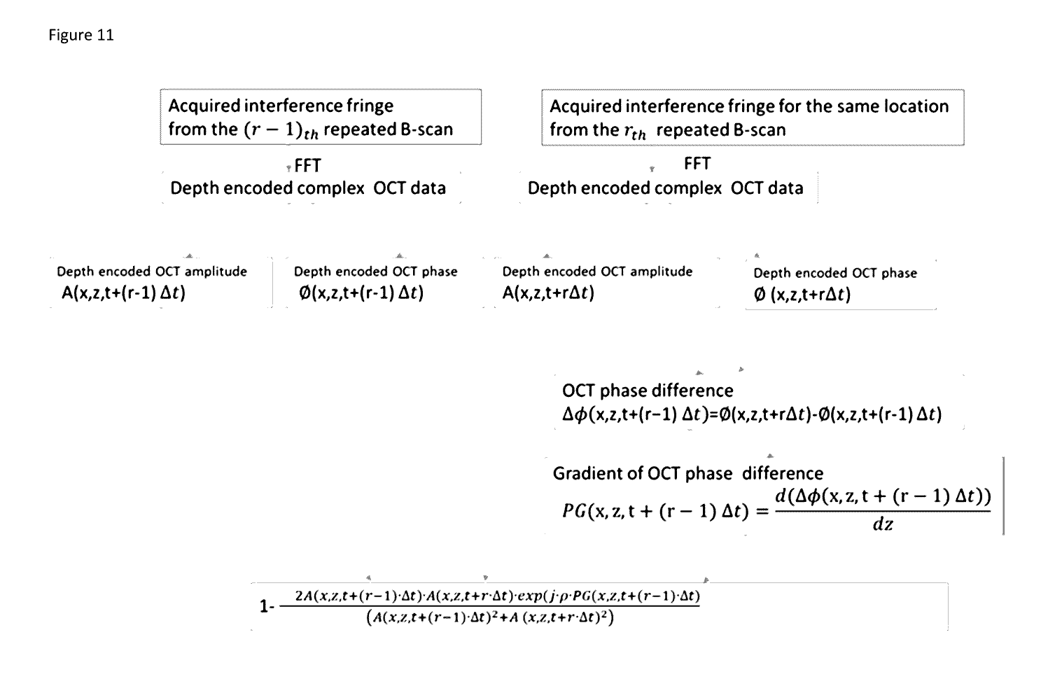

Amplitude/intensity OCTA methods based on the difference, variance, or decorrelation of amplitude/intensity signal have been demonstrated (Mariampillai et al 2008 supra; Liu G et al 2011 supra; and Blatter C et al, J Biomed Opt 17, 0705051-0705054 (2012); incorporated by reference herein). Since decorrelation based algorithms have a normalized value, a decorrelation algorithm to combine the phase gradient contrast with amplitude/intensity contrast can be expressed as shown in Equation 16 below.

$${C}_{f} ( x , z ) = 1 - \frac{1}{R - 1} \vert {\sum }_{r = 1}^{R - 1} \frac{\begin{matrix}2 A ( \begin{matrix}x , z , t + \\ ( r - 1 ) \cdot Δ t\end{matrix} ) \cdot \\ A ( x , z , t + r \cdot Δ t ) \cdot \\ exp ( \begin{matrix}j \cdot ρ \cdot \\ PG ( \begin{matrix}x , z , t + \\ ( r - 1 ) \cdot Δ t\end{matrix} )\end{matrix}\end{matrix}}{\begin{matrix}{A ( \begin{matrix}x , z , t + \\ ( r - 1 ) \cdot Δ t\end{matrix} )}^{2} + \\ {A ( x , z , t + r \cdot Δ t )}^{2}\end{matrix}} \vert \qquad Equation 16$$

where C_{f} is the flow contrast, R is the number of scan repetitions at the same B-scan location, and ρ is a weight parameter that controls the contribution from phase gradient contrast. In the examples that follow, ρ=2 was used when applying Equation 16. FIG. 11 shows the flow chart to calculate the flow contrast from the Equation 16. Depth encoded complex OCT data are obtained through Fourier transform of the acquired fringes. The phase difference between A-scans at the same location in repeated B-scans is obtained. The gradient of the phase difference is calculated and then combined with amplitude to calculate the flow contrast.

Split spectrum methods have been shown to improve the SNR of flow detection (Jia Y et al 2012 supra; Gao S S et al, Opt Letters 40, 2305-2308 (2015); incorporated by reference herein). After splitting the spectrum into several narrow bands, the data in each narrower bands are processed separately to generate several OCTA images for the same B-scan location. These images are then averaged to achieve a higher SNR image for the specific B-scan location. A method to calculate SSPGA is shown in Equation 17 below.

$$SSPGA ( x , z ) = \frac{1}{R - 1} \frac{1}{M} {\sum }_{m = 1}^{M} {\sum }_{r = 1}^{R - 1} \vert {PG}_{m} ( x , z , t + ( r - 1 ) \cdot Δ t ) \vert \qquad Equation 17$$

where M is the number of narrow split spectrum bands, and R is the number of scan repetitions at the same B-scan location.

A method to calculate SSAPGA is shown in Equation 18 below.

$$SSAPGA ( x , z ) = 1 - \frac{1}{R - 1} \frac{1}{M} \vert {\sum }_{m = 1}^{M} {\sum }_{r = 1}^{R - 1} \frac{\begin{matrix}2 {A}_{m} ( x , z , t + ( r - 1 ) \cdot Δ t ) \cdot \\ {A}_{m} ( x , z , t + r \cdot Δ t ) \cdot \\ exp ( \begin{matrix}j \cdot ρ \cdot \\ {PG}_{m} ( x , z , t + ( r - 1 ) \cdot Δ t )\end{matrix}\end{matrix}}{\begin{matrix}{[ {A}_{m} ( \begin{matrix}x , z , t + \\ ( r - 1 ) \cdot Δ t\end{matrix} ) ]}^{2} + \\ {[ {A}_{m} ( x , z , t + r \cdot Δ t ) ]}^{2}\end{matrix}} \vert \qquad Equation 18$$

where M is the number of narrow split spectrum bands, and R is the number of scan repetitions at the same B-scan location.

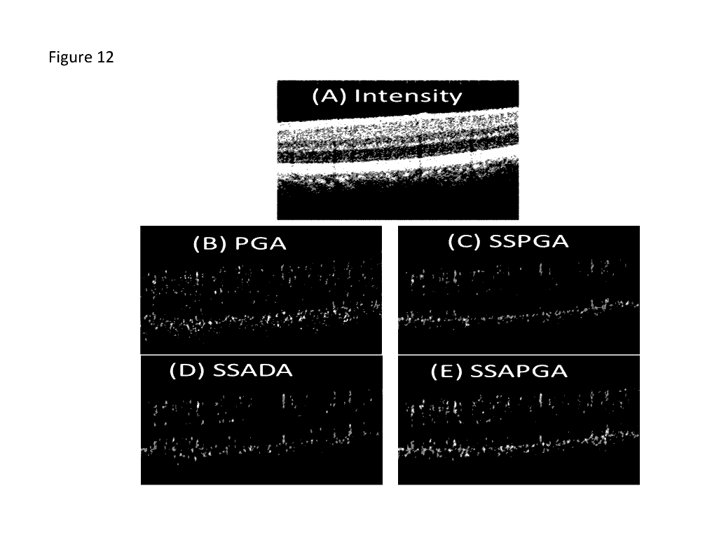

Results using a spectral domain OCT system: The algorithm was tested for clinical retinal imaging (FIG. 12) on a healthy human participant using a 70 kHz, 840 nm spectral domain OCT system (RTVue-XR, Optovue). The imaging protocol consisted of a single volumetric scan covering a 3×3 mm area centered at the fovea. In the fast transverse scanning direction, 304 A-scans were sampled to obtain a single B-scan. For flow detection, two sequential B-scans were collected at each location. In the slow transverse scanning direction, 304 locations were sampled. The total imaging time was approximately 3 seconds.

Two different split-spectrum PGA methods (SSPGA, and SSAPGA), each with 11 split-spectrum bands, were used to process the data. The acquired spectrum was split into eleven different narrow band spectrums using Gaussian windows with different central wavelengths but the same full width of half maximum value. The 11 narrow band spectrums were processed separately, using typical SD-OCT data processing procedures including numerical interpolation for wavelength calibration, numerical dispersion compensation, and fast Fourier transform (FFT). The phase term of the complex OCT signal (obtained after the FFT operation) was used to obtain the PGA according to Equation 15. The PGA signals from different bands were averaged to get the SSPGA image according to Equation 17. The PGA signals were also combined with the amplitude signal in each band and further averaged according to Equation 18 to get the SSAPGA image. Results from PGA (non-split-spectrum) and SSADA (11 split bands) were also obtained for comparison. All the OCTA images were thresholded with a value equal to sum of the median OCTA and 3 times standard deviation in the retinal tissue region.

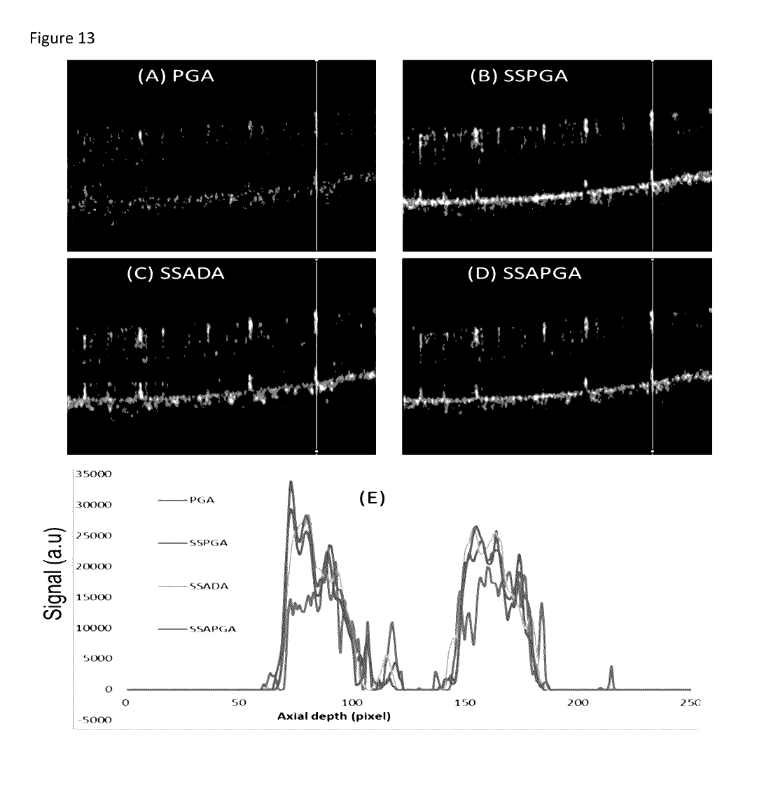

Cross-sectional OCT and OCTA images of a health eye are shown in FIG. 12. All the images are processed from the same dataset. The images from the three split-spectrum methods (SSPGA, SSADA and SSAPGA) do not show major difference (FIGS. 12B-E). The PGA method visually shows higher noise, which manifests as random dots in the image (FIG. 12A). By averaging 5 adjacent frames of cross-sectional OCTA images (FIGS. 13A-D), noise in the OCTA image can be further reduced and single OCTA A-scan profiles (FIG. 13E) from different methods can be extracted and compared. Methods using the split-spectrum technique show much more smoothed profiles. This is due to lower noise and decreased resolution that results from application of the split-spectrum method. The PGA method shows overall higher noise. SSADA (FIG. 13C) shows more signal at the inner segment-outer segment (IS-OS) junctional layer and at the retina pigment epithelium (RPE) layer. This suggests that SSADA is more prone to vessel projection artifacts than the other methods.

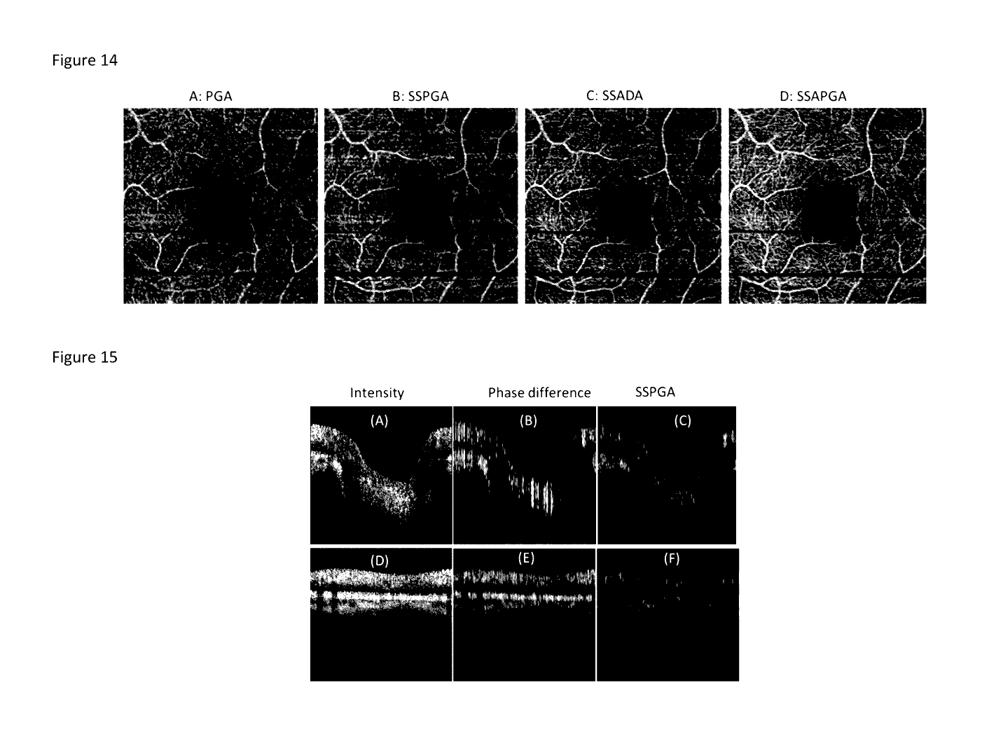

The performance of these methods was also compared in en-face view, which is the preferred way to evaluate retinal microvasculature clinically (FIG. 14). The 3D image data was separated into retinal and choroidal regions with the dividing boundary set at the RPE layer. FIG. 14 shows the average intensity projection angiography images for human retinal vasculature from PGA (FIG. 14A), SSPGA (FIG. 14B), SSADA (FIG. 14C) and SSAPGA (FIG. 14D). Qualitatively, the images exhibit very similar patterns of retinal vasculature. Quantitative comparisons were made using two different parameters: the image signal-to-noise ratio (SNR) SNR and the image contrast. SNR for flow detection was calculated as shown in Equation 19 below.

$$SNR = \frac{average of the signal region}{standard deviation of the noise region} \qquad Equation 19$$

The foveal avascular zone was taken as the noise baseline. Root mean square (RMS) contrast was used to characterize image contrast, and is defined as the standard deviation of the pixel intensities as shown in Equation 20 below.

$${C}_{rms} = \sqrt{\frac{1}{XY} {\sum }_{x = 1}^{X} {\sum }_{y = 1}^{Y} {[ V ( x , y ) - \overline{V} ]}^{2}} \qquad Equation 20$$

where X,Y is the number of pixels in the two dimensional image, V(x,y) is the angiography value for the pixel at the (x,y) location in the image and V̅ is the average value of all pixels in the image. Quantitative comparison results are shown in Table 1, with the PGA image set as reference. Compared to PGA (FIG. 14A), SNR increased by 129.04%, 267.0%, and 355.9% for SSPGA (FIG. 14B), SSADA (FIG. 14C) and SSAPGA (FIG. 14D), respectively. Among the three split spectrum methods, SSAPGA shows best image contrast and SNR and SSPGA shows the worst image contrast and SNR.

TABLE 1
Quantitative comparison of different OCTA methods

| Method | SNR | Improvement | Contrast | Improvement |
|---|---|---|---|---|
| PGA | 4.06 | — | 37.6 | — |
| SSPGA | 9.3 | 129.04% | 40.54 | 7.8% |
| SSADA | 14.9 | 267.0% | 45.89 | 22.0% |
| SSAPGA | 18.51 | 355.9% | 78.67 | 109.2% |

Results using a Swept source OCT system: A custom-built SS-OCT system was used to test the algorithms. Briefly, the system used a 200 kHz swept laser with a central wavelength of 1050 nm and bandwidth of 100 nm (Axsun 1050). The system was based on a fiber Mach-Zehnder interferometer. The final interference signal is detected by a balanced detector and digitized by a high speed digitizer (ATS9360, Alazar Technologies). Each A-scan was comprised of 1216 data points. The laser power on the cornea is 1.4 mW with a beam spot size approximately 2 mm. The imaging protocol consisted of a single volumetric scan covering a 3×3 mm area of the disk in a healthy human participant. In the fast transverse scanning direction, 600 A-scans were sampled to obtain a single B-scan (100 A-scans for galvonometer flyback). Three sequential B-scans were collected at each location for flow detection. In the slow transverse scanning direction, 500 locations were sampled. The data processing steps were similar to those used for the spectral domain system described above, and 11 sub-bands were used for split-spectrum calculation.

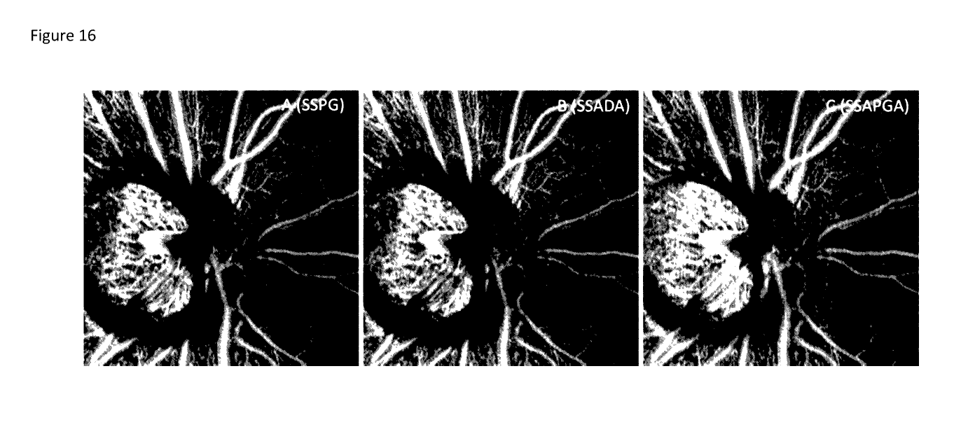

FIG. 15 shows the both cross sectional structure OCT and OCTA images from a healthy eye acquired with the SS-OCT system. Due to the phase instability of the SS-OCT system, the phase difference image (FIGS. 15B and 15E) shows strong vertical line artifacts. By taking the axial gradient of the phase difference, the result show clearer vessel boundaries (FIGS. 15C and 15F). It should be noted that no phase instability correction or bulk phase removal method was used to process these images. FIG. 16 shows the en-face projection OCTA images from SSPGA, SSADA, SSAPGA methods, respectively. The three split-spectrum methods showed similar results, with the SSAPGA having the overall best qualitative performance.

### Example 4

### Optical Coherence Tomography Angiography Image Processing System

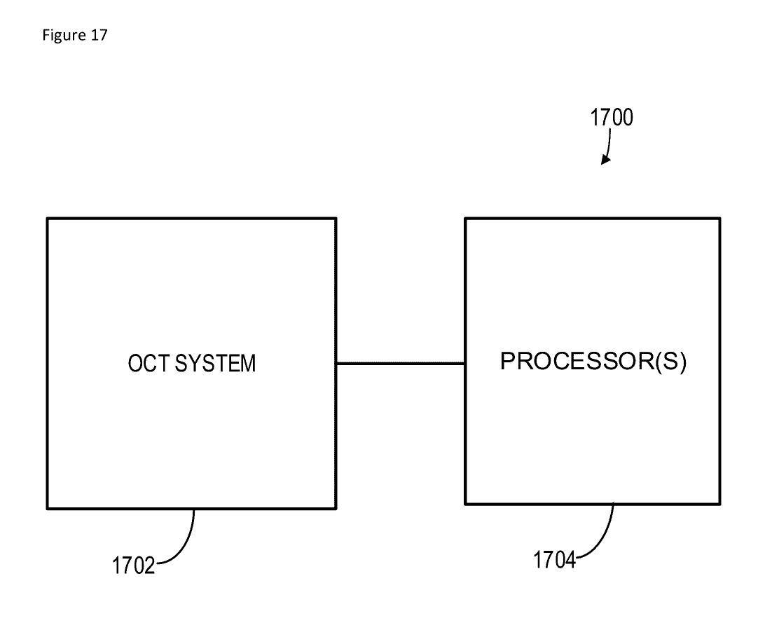

FIG. 17 schematically shows an example system 1700 for OCT image processing in accordance with various embodiments. System 1700 comprises an OCT system 1702 configured to acquire an OCT image comprising OCT interferograms and one or more processors or computing systems 1704 that are configured to implement the various processing routines described herein. OCT system 1700 can comprise an OCT system suitable for OCT angiography applications, e.g., a swept source OCT system or spectral domain OCT system.

In various embodiments, an OCT system can be adapted to allow an operator to perform various tasks. For example, an OCT system can be adapted to allow an operator to configure and/or launch various ones of the herein described methods. In some embodiments, an OCT system can be adapted to generate, or cause to be generated, reports of various information including, for example, reports of the results of scans run on a sample.

In embodiments of OCT systems comprising a display device, data and/or other information can be displayed for an operator. In embodiments, a display device can be adapted to receive an input (e.g., by a touch screen, actuation of an icon, manipulation of an input device such as a joystick or knob, etc.) and the input can, in some cases, be communicated (actively and/or passively) to one or more processors. In various embodiments, data and/or information can be displayed, and an operator can input information in response thereto.

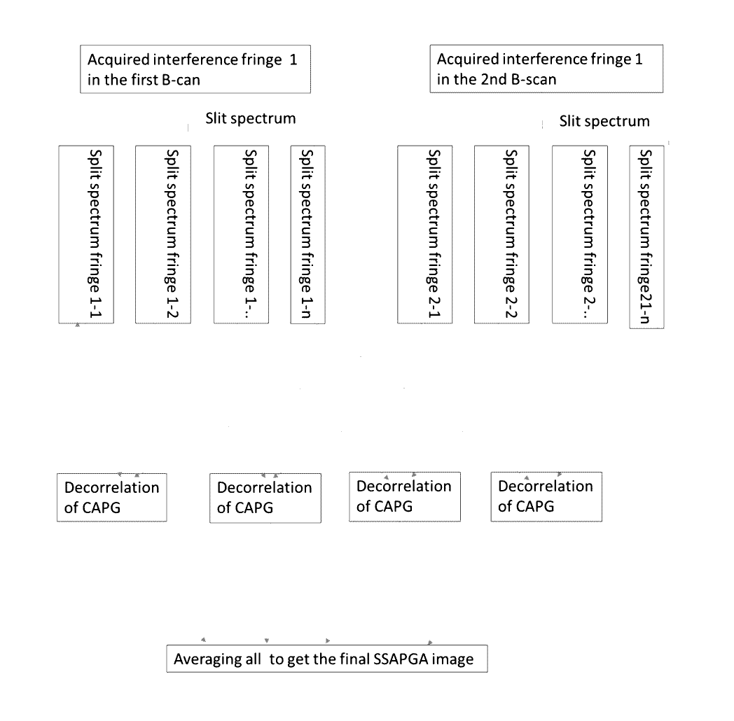

In some embodiments, the above described methods and processes can be tied to a computing system, including one or more computers. In particular, the methods and processes described herein, e.g., the methods depicted in FIGS. 1-5 and FIG. 11 described above, can be implemented as a computer application, computer service, computer API, computer library, and/or other computer program product.

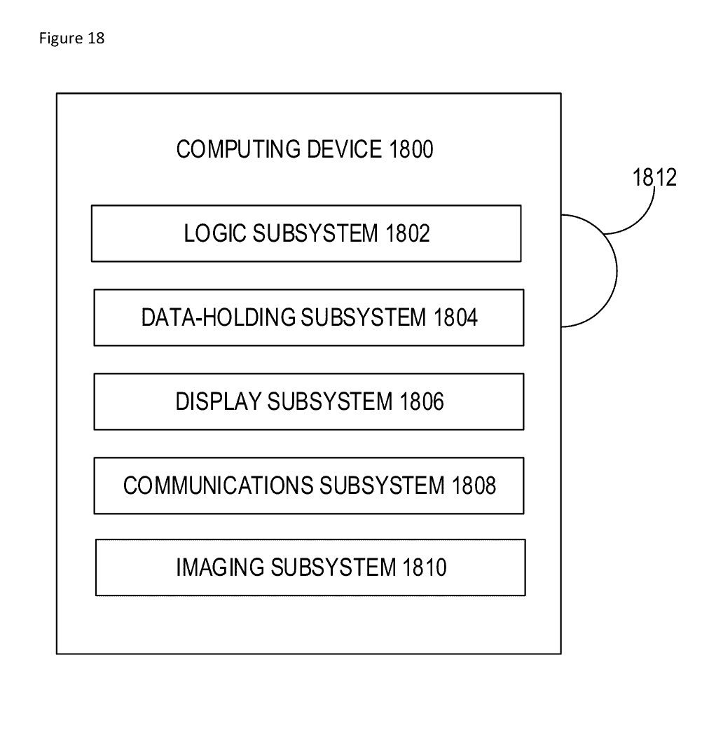

FIG. 18 schematically shows a non-limiting computing device 1800 that can perform one or more of the above described methods and processes. For example, computing device 1800 can represent a processor included in system 1700 described above, and can be operatively coupled to, in communication with, or included in an OCT system or OCT image acquisition apparatus. Computing device 1800 is shown in simplified form. It is to be understood that virtually any computer architecture can be used without departing from the scope of this disclosure. In different embodiments, computing device 1800 can take the form of a microcomputer, an integrated computer circuit, printed circuit board (PCB), microchip, a mainframe computer, server computer, desktop computer, laptop computer, tablet computer, home entertainment computer, network computing device, mobile computing device, mobile communication device, gaming device, etc.

Computing device 1800 includes a logic subsystem 1802 and a data-holding subsystem 1804. Computing device 1800 can optionally include a display subsystem 1806, a communication subsystem 1808, an imaging subsystem 1810, and/or other components not shown in FIG. 18. Computing device 1800 can also optionally include user input devices such as manually actuated buttons, switches, keyboards, mice, game controllers, cameras, microphones, and/or touch screens, for example.

Logic subsystem 1802 can include one or more physical devices configured to execute one or more machine-readable instructions. For example, the logic subsystem can be configured to execute one or more instructions that are part of one or more applications, services, programs, routines, libraries, objects, components, data structures, or other logical constructs. Such instructions can be implemented to perform a task, implement a data type, transform the state of one or more devices, or otherwise arrive at a desired result.

The logic subsystem can include one or more processors that are configured to execute software instructions. For example, the one or more processors can comprise physical circuitry programmed to perform various acts described herein. Additionally or alternatively, the logic subsystem can include one or more hardware or firmware logic machines configured to execute hardware or firmware instructions. Processors of the logic subsystem can be single core or multicore, and the programs executed thereon can be configured for parallel or distributed processing. The logic subsystem can optionally include individual components that are distributed throughout two or more devices, which can be remotely located and/or configured for coordinated processing. One or more aspects of the logic subsystem can be virtualized and executed by remotely accessible networked computing devices configured in a cloud computing configuration.

Data-holding subsystem 1804 can include one or more physical, non-transitory, devices configured to hold data and/or instructions executable by the logic subsystem to implement the herein described methods and processes. When such methods and processes are implemented, the state of data-holding subsystem 1804 can be transformed (e.g., to hold different data).

Data-holding subsystem 1804 can include removable media and/or built-in devices. Data-holding subsystem 1804 can include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1804 can include devices with one or more of the following characteristics: volatile, nonvolatile, dynamic, static, read/write, read-only, random access, sequential access, location addressable, file addressable, and content addressable. In some embodiments, logic subsystem 1802 and data-holding subsystem 1804 can be integrated into one or more common devices, such as an application specific integrated circuit or a system on a chip.

FIG. 18 also shows an aspect of the data-holding subsystem in the form of removable computer-readable storage media 1812, which can be used to store and/or transfer data and/or instructions executable to implement the herein described methods and processes. Removable computer-readable storage media 1812 can take the form of CDs, DVDs, HD-DVDs, Blu-Ray Discs, EEPROMs, flash memory cards, USB storage devices, and/or floppy disks, among others.

When included, display subsystem 1806 can be used to present a visual representation of data held by data-holding subsystem 1804. As the herein described methods and processes change the data held by the data-holding subsystem, and thus transform the state of the data-holding subsystem, the state of display subsystem 1806 can likewise be transformed to visually represent changes in the underlying data. Display subsystem 1806 can include one or more display devices utilizing virtually any type of technology. Such display devices can be combined with logic subsystem 1802 and/or data-holding subsystem 1804 in a shared enclosure, or such display devices can be peripheral display devices.

When included, communication subsystem 1808 can be configured to communicatively couple computing device 1800 with one or more other computing devices. Communication subsystem 1808 can include wired and/or wireless communication devices compatible with one or more different communication protocols. As non-limiting examples, the communication subsystem can be configured for communication via a wireless telephone network, a wireless local area network, a wired local area network, a wireless wide area network, a wired wide area network, etc. In some embodiments, the communication subsystem can allow computing device 1800 to send and/or receive messages to and/or from other devices via a network such as the Internet.

When included, imaging subsystem 1810 can be used acquire and/or process any suitable image data from various sensors or imaging devices in communication with computing device 1800. For example, imaging subsystem 1810 can be configured to acquire OCT image data, e.g., interferograms, as part of an OCT system, e.g., OCT system 1702 described above. Imaging subsystem 1810 can be combined with logic subsystem 1802 and/or data-holding subsystem 1804 in a shared enclosure, or such imaging subsystems can comprise periphery imaging devices. Data received from the imaging subsystem can be held by data-holding subsystem 1804 and/or removable computer-readable storage media 1812, for example.

It is to be understood that the configurations and/or approaches described herein are exemplary in nature, and that these specific embodiments or examples are not to be considered in a limiting sense, because numerous variations are possible. The specific routines or methods described herein can represent one or more of any number of processing strategies. As such, various acts illustrated can be performed in the sequence illustrated, in other sequences, in parallel, or in some cases omitted. Likewise, the order of the above-described processes can be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A computer-based method of imaging vascular flow using optical coherence tomography (OCT), the method comprising:
acquiring a first interference fringe from a first B-scan and a second interference fringe from a second B-scan;
transforming the first interference fringe into a first set of depth encoded OCT data;
transforming the second interference fringe into a second set of depth encoded OCT data;
acquiring a first set of depth encoded OCT phase values from the first set of depth encoded OCT data;
acquiring a second set depth encoded OCT phase values from the second set of depth encoded OCT data;
calculating a set of OCT phase difference values using the first set of depth encoded OCT phase values and the second set of depth encoded OCT phase values;
calculating a set of OCT phase gradient values from the set of OCT phase difference values.

2. The method of claim 1 comprising using a fast Fourier transform to transform the first interference fringe into the first set of depth encoded OCT data and to transform the second interference fringe into the second set of depth encoded OCT data.

3. The method of claim 1 comprising calculating the set of OCT phase gradient values using Equation 4.

4. The method of claim 1 further comprising acquiring a first set of depth encoded OCT amplitude values from the first set of depth encoded OCT data; and calculating a first combination signal from the first set of depth encoded OCT amplitude values and the set of OCT phase gradient values.

5. The method of claim 4 further comprising calculating the first combination signal using Equation 5.

6. The method of claim 1 further comprising acquiring a third interference fringe from a third B-scan; transforming the third interference fringe into a third set of depth encoded OCT data; acquiring a third set of depth encoded OCT phase values; acquiring a second set of depth encoded OCT amplitude values; calculating a second set of OCT phase difference values from the second set of depth encoded OCT phase values and the third set of depth encoded OCT phase values; calculating a second set of OCT phase gradient values from the second set of OCT phase difference values; calculating a second combination signal from the second set of depth encoded OCT amplitude values and the second set of OCT phase gradient values; and calculating a first decorrelation from the first combination signal and the second combination signal.

7. The method of claim 6 further comprising calculating the first decorrelation using Equation 6.

8. The method of claim 6 further comprising calculating the first decorrelation using Equation 16.

9. The method of claim 6 further comprising calculating a second decorrelation and averaging the first decorrelation and the second decorrelation.

10. The method of claim 1 further comprising imaging vascular flow using swept source optical coherence tomography.

11. A computer-based method of imaging vascular flow using optical coherence tomography (OCT), the method comprising:
splitting the spectrum of a first B-scan into M spectral bands;
splitting the spectrum of a second B-scan into M spectral bands; calculating M sets of phase gradient values according to claim 1; and
calculating a single set of OCT phase gradient values from the M sets of phase gradient values;

12. The method of claim 11 wherein calculating a single set of OCT phase gradient values comprises averaging the M sets of phase gradient values.

13. The method of claim 11 comprising calculating the single set of OCT phase gradient values using Equation 17.

14. The method of claim 11, wherein splitting the spectrum of a B-scan into M spectral bands comprises:
creating overlapping filters covering the OCT spectrum; and
filtering the OCT spectrum with the overlapping filters.

15. The method of claim 14, wherein creating overlapping filters comprises creating a filter bank comprised of at least one specification.

16. The method of claim 15, wherein the at least one specification is comprised of one or more factors comprising at least one of a filter type, a bandwidth of a filter, an overlap between different bands, and a number of bands.

17. The method of claim 11 further comprising: calculating M sets of combination signals according to claim 4; and
calculating a single combination signal from the M sets of combination signals;

18. The method of claim 17 comprising calculating the single combination signal using Equation 18.

19. The method of claim 11 further comprising imaging vascular flow using swept source optical coherence tomography.
